(19) United States
(12) Patent Application Publication
(10) Pub. No.: US 2024/0229072 A9
(43) Pub. Date: Jul. 11, 2024

(54) VIRAL VECTOR-BASED GENE THERAPY FOR OCULAR CONDITIONS

(71) Applicant: The Regents of the University of Colorado, A Body Corporate, Denver, CO (US)

(72) Inventors: Ram H. NAGARAJ, Aurora, CO (US); Rooban B. NAHOMI, Aurora, CO (US)

(21) Appl. No.: 18/547,348

(22) PCT Filed: Feb. 22, 2022
(86) PCT No.: PCT/US2022/017279

§ 371 Date: Aug. 22, 2023

Related U.S. Application Data
(60) Provisional application No. 63/152,152, filed on Feb. 22, 2021.

(51) Int. Cl. C12N 15/86; A61K 9/00; A61K 48/00; A61P 27/06; C07K 14/47

(52) U.S. Cl. CPC C12N 15/86; A61K 9/0048; A61K 48/0075; A61P 27/06; C07K 14/47; C12N 2750/14143

(57) ABSTRACT

Gene therapy for a retinal disease, injury, or condition in a subject involves administering to the subject a pharmaceutical composition containing a recombinant adeno-associated viral vector encoding at least one heat shock protein, such as Hsp27. A recombinant adeno-associated viral vector can include a promoter sequence that induces production of a heat shock protein specifically in retinal ganglion cells. The loss of such cells causes retinal damage and loss of eyesight in patients afflicted with an ocular condition. The disclosed viral vector may be included in pharmaceutical compositions that may be administered intravitreally using an administration device. A single injection 10 may be therapeutically sufficient for treating various ocular conditions.

### CROSS-REFERENCE TO RELATED APPLICATION

This application claims priority to U.S. Provisional Patent Application No. 63/152,152, filed Feb. 22, 2021, entitled “Viral Vector-Based Gene Therapy for Ocular Conditions,” which is incorporated by reference herein, in the entirety and for all purposes.

### STATEMENT OF GOVERNMENT INTEREST

This invention was made with support from the National Eye Institute, along with additional support from the Gates Center for Regenerative Medicine, as well as Research to Prevent Blindness. The government has certain rights in the invention.

### TECHNICAL FIELD

The present disclosure relates generally to compositions, systems, and methods for treating retinal damage caused by injury or disease. Specific implementations involve viral vector-mediated delivery of at least one heat shock protein to the retinal ganglion cells of a subject afflicted with, or at risk of developing, ocular damage.

### BACKGROUND

Glaucoma affects nearly 75 million people worldwide, and approximately 8 million people are blind from the disease. Nearly 3 million people are afflicted with glaucoma in the United States alone, and this number is expected to more than double by 2050. Because glaucoma-associated vision loss is often attributed largely to elevated pressure inside the eye, known as intraocular pressure, the conventional first line of glaucoma treatment usually involves topical application of drugs formulated to lower intraocular pressure. Even if this approach successfully lowers the pressure, however, many patients still go blind because of axonal degeneration and the continued death of cells in the retina, known as retinal ganglion cells (“RGCs”). The diversity of factors contributing to axonal degeneration and RGC death, both individually and especially in combination, make glaucoma and other ocular conditions difficult to treat. Accordingly, safe and effective methods of combating RGC death and axonal degeneration are needed.

### SUMMARY

The present disclosure includes novel gene therapies for various ocular conditions, including glaucoma. Embodiments include recombinant adeno-associated viral vectors (“rAAV vectors”) that contain at least one nucleic acid sequence encoding at least one heat shock protein (“HSP”). The disclosed rAAV vectors can also include an RGC-specific promoter sequence that induces targeted expression of the encoded HSPs where they are needed most in the eye. Successful treatment, prevention, and/or alleviation of at least one symptom of an ocular condition caused by retinal damage can be achieved via a single administration of the rAAV-HSP vectors. As illustrated by the experimental data summarized herein, the disclosed vectors, pharmaceutical compositions, and associated therapies may prevent or treat retinal damage by substantially blocking, slowing and/or reducing RGC death over prolonged periods of time (e.g., at least about 20 weeks).

In accordance with specific embodiments of the present disclosure, a method of treating, reducing the risk of, preventing, and/or alleviating at least one symptom of a retinal disease, injury, or condition in a subject may involve administering to the subject a therapeutically effective amount of a composition comprising an rAAV vector. The rAAV vector can include a nucleic acid sequence encoding at least one biologically active heat shock protein, such as Hsp27 (also referred to herein as “HspB1”). The rAAV vector can also include a promoter sequence positioned upstream of the nucleic acid sequence. The promoter sequence can induce expression of the nucleic acid sequence in retinal ganglion cells.

In some embodiments of the method, the retinal ganglion cells can comprise mammalian retinal ganglion cells. In some embodiments of the method, the mammalian retinal ganglion cells can comprise human retinal ganglion cells. In some embodiments of the method, the composition can be administered at least once within 24 hours after an ocular injury is sustained by a subject or a retinal disease or condition is diagnosed in the subject. In some embodiments of the method, the composition can be administered intravitreally. In some embodiments, the composition can be administered only once. In some embodiments of the method, the rAAV vector can be an adeno-associated virus-Type 2 vector.

In some embodiments of the method, the retinal disease, injury, or condition is glaucoma. In some embodiments of the method, the retinal disease, injury, or condition is selected from the group consisting of: macular degeneration, diabetic eye disease, retinal detachment, and retinitis pigmentosa. In some embodiments of the method, the retinal disease, injury, or condition is caused by excitotoxic damage, physical damage, chemical damage, neurotrophic factor deprivation, oxidative stress, inflammation, mitochondrial dysfunction, axonal transport failure, or combinations thereof. In some embodiments of the method, the retinal disease, injury, or condition comprises a loss of retinal ganglion cells. In some embodiments of the method, the retinal disease, injury, or condition comprises an increase in intraocular pressure.

In accordance with embodiments of the present disclosure, a method of increasing Hsp27 protein production in the retinal ganglion cells of a subject involves administering to an eye of the subject a therapeutically effective amount of a composition comprising an rAAV vector. The rAAV vector can include a nucleic acid sequence encoding the Hsp27 protein, along with a promoter sequence positioned upstream of the nucleic acid sequence. The promoter sequence can induce expression of the nucleic acid sequence in the retinal ganglion cells. The amount of Hsp27 protein can be increased in the retinal ganglion cells of the treated eye compared to retinal ganglion cells of another eye to which the composition is not administered. In some embodiments of the method, the retinal ganglion cells of the subject can comprise human retinal ganglion cells. In some embodiments of the method, the rAAV vector can be an adeno-associated virus-Type 2 vector.

In accordance with embodiments of the present disclosure, a system for treating, reducing the risk of, preventing, and/or alleviating at least one symptom of a retinal disease, injury, or condition in a subject may include an injection device and a therapeutically effective amount of a composition comprising an rAAV vector. The rAAV vector can include a nucleic acid sequence encoding at least one biologically active heat shock protein, such as Hsp27. The rAAV vector can also include a promoter sequence positioned upstream of the nucleic acid sequence. The promoter sequence can induce expression of the nucleic acid sequence in retinal ganglion cells. The injection device can be configured to administer the composition to the subject intravitreally.

In some embodiments of the system, the injection device can be a tuberculin syringe. In some embodiments of the system, the retinal disease, injury, or condition is glaucoma. In some embodiments of the system, the retinal disease, injury, or condition comprises a loss of retinal ganglion cells. In some embodiments of the system, the retinal disease, injury, or condition comprises an increase in intraocular pressure. In some embodiments of the system, the retinal ganglion cells can comprise mammalian retinal ganglion cells. In some embodiments of the system, the retinal ganglion cells can comprise human retinal ganglion cells. In some embodiments of the system, the injection device can be a single-use device. In some embodiments of the system, the rAAV vector can be an adeno-associated virus-Type 2 vector.

In accordance with embodiments of the present disclosure, a pharmaceutical composition can include an rAAV vector and a pharmaceutically acceptable carrier. The rAAV vector can include a nucleic acid sequence encoding at least one biologically active heat shock protein, such as Hsp27. The rAAV vector can also include a promoter sequence positioned upstream of the nucleic acid sequence. The promoter sequence can induce expression of the nucleic acid sequence in retinal ganglion cells. The pharmaceutical composition can be formulated for treating, reducing the risk of, preventing, or alleviating at least one symptom of a retinal disease, injury, or condition in a subject.

In some embodiments of the composition, the retinal ganglion cells can comprise mammalian retinal ganglion cells. In some embodiments of the composition, the mammalian retinal ganglion cells can comprise human retinal ganglion cells. In some embodiments of the composition, the pharmaceutical composition can be formulated for intravitreal administration. In some embodiments of the composition, the rAAV vector can be an adeno-associated virus-Type 2 vector. In some embodiments of the composition, the retinal disease, injury, or condition comprises a loss of retinal ganglion cells, an increase in intraocular pressure, and/or glaucoma.

In accordance with embodiments of the present disclosure, a pharmaceutical composition comprising an rAAV vector can be used in the manufacture of a medicament for treating, reducing the risk of, preventing, or alleviating at least one symptom of a retinal disease, injury, or condition in a subject. The rAAV vector can include include a nucleic acid sequence encoding at least one biologically active heat shock protein, such as Hsp27. The rAAV vector can also include a promoter sequence positioned upstream of the nucleic acid sequence. The promoter sequence can induce expression of the nucleic acid sequence in retinal ganglion cells.

In some manufacturing embodiments, the pharmaceutical composition can be formulated for intravitreal administration. In some manufacturing embodiments, the retinal disease, injury, or condition comprises glaucoma. In some manufacturing embodiments, the retinal disease, injury, or condition comprises a loss of retinal ganglion cells. In some manufacturing embodiments, the retinal disease, injury, or condition comprises an increase in intraocular pressure.

In accordance with embodiments of the present disclosure, an rAAV vector can include a nucleic acid sequence encoding at least one biologically active heat shock protein, such as Hsp27. The rAAV vector can also include a promoter sequence positioned upstream of the nucleic acid sequence. The promoter sequence can induce expression of the nucleic acid sequence in retinal ganglion cells. The rAAV vector can be formulated for treating, reducing the risk of, preventing, or alleviating at least one symptom of a retinal disease, injury, or condition in a subject.

This Summary is neither intended as, nor should it be construed as, being representative of the full extent and scope of the present disclosure. Moreover, references made herein to “the present disclosure,” or aspects thereof, should be understood to mean certain embodiments of the present disclosure and should not necessarily be construed as limiting all embodiments to a particular description. The present disclosure is set forth in various levels of detail in this Summary as well as in the attached drawings and Detailed Description, and no limitation as to the scope of the present disclosure is intended by either the inclusion or non-inclusion of elements, components, etc. in this Summary. Features from any of the disclosed embodiments may be used in combination with one another, without limitation. In addition, other features and advantages of the present disclosure will become apparent to those of ordinary skill in the art through consideration of the following Detailed Description and the accompanying drawings.

### BRIEF DESCRIPTION OF THE DRAWINGS

The drawings illustrate several embodiments of the invention, wherein identical reference numerals refer to identical or similar elements or features in different views or embodiments shown in the drawings.

FIG. 1 is a map of an rAAV vector that includes a nucleic acid sequence encoding the Hsp27 protein according to embodiments disclosed herein.

FIG. 2 is a map of an rAAV vector that includes a nucleic acid sequence encoding the αA-crystallin protein according to embodiments disclosed herein.

FIG. 3 is a map of an rAAV vector that includes a nucleic acid sequence encoding the αB-crystallin protein according to embodiments disclosed herein.

FIG. 4 is a map of an rAAV vector that includes a nucleic acid sequence encoding the Hsp20 protein according to embodiments disclosed herein.

FIG. 5 is a confocal microscopy image showing the effects of various rAAV2 vectors on RGCs derived from healthy and glaucomatous mice via Brna3-immunostaining according to embodiments disclosed herein.

FIG. 6 is a bar graph showing the quantitative effects of various rAAV2 vectors on RGCs derived from healthy and glaucomatous mice according to embodiments disclosed herein.

FIG. 7A is a confocal microscopy image showing the expression of Hsp27 in RGCs mediated by intravitreal administration of rAAV2-Hsp27. FIG. 7B is a Western blot showing the Hsp27 protein extracted from the RGCs depicted in FIG. 7A.

FIG. 8A is a confocal microscopy image showing the expression of αA-crystallin in RGCs mediated by intravitreal administration of rAAV2-αA-crystallin. FIG. 8B is a Western blot showing the αA-crystallin protein extracted from the RGCs depicted in FIG. 8A.

FIG. 9A is a confocal microscopy image showing the expression of αB-crystallin in RGCs mediated by intravitreal administration of rAAV2-αB-crystallin. FIG. 9B is a Western blot showing the αB-crystallin protein extracted from the RGCs depicted in FIG. 9A.

FIG. 10A is a confocal microscopy image showing the expression of Hsp20 in RGCs mediated by intravitreal administration of rAAV2-Hsp20. FIG. 10B is a Western blot showing the Hsp20 protein extracted from the RGCs depicted in FIG. 10A.

FIG. 11A is a line graph showing the effects of microbead injection on intraocular pressure according to embodiments disclosed herein.

FIG. 11B is a bar graph showing the effects of intravitreal rAAV2-HspB1 administration on RGC death in the microbead-based mouse model of ocular hypertension represented in FIG. 11A.

FIG. 11C is a confocal microscopy image showing the effects of intravitreal rAAV2-HspB1 administration on RGCs of healthy mice, or mice afflicted with ocular hypertension, using Brna3 immunostaining according to embodiments disclosed herein.

FIG. 12A is a bar graph showing the effects of intravitreal rAAV2-HspB1 administration on RGC axonal transport in a microbead-based mouse model of ocular hypertension according to embodiments disclosed herein.

FIG. 12B is a confocal microscopy image showing the effects of intravitreal rAAV2-HspB1 administration on RGC axonal transport using CT-B staining according to embodiments disclosed herein.

FIG. 13A is a line graph showing the effects of multiple microbead injections on intraocular pressure before and after rAAV2-HspB1 administration according to embodiments disclosed herein.

FIG. 13B is a line graph showing the effects of intravitreal rAAV2-HspB1 administration on RGC death in the microbead-based mouse model of ocular hypertension represented in FIG. 13A.

FIG. 13C is a confocal microscopy image showing the effects of intravitreal rAAV2-HspB1 administration on RGCs represented in FIG. 13B using Brna3 immunostaining according to embodiments disclosed herein.

FIG. 14A is a bar graph showing the effects of intravitreal rAAV2-HspB1 administration on RGC axonal transport in a microbead-based mouse model of ocular hypertension.

FIG. 14B is a confocal microscopy image showing the effects of intravitreal rAAV2-HspB1 administration on RGC axonal transport represented in FIG. 14A using CT-B staining.

FIG. 15A is a line graph showing the effects of microbead injection on intraocular pressure for a 20-week period after rAAV2-HspB1 administration according to embodiments disclosed herein.

FIG. 15B is a bar graph showing pattern electroretinogram (“PERG”) amplitudes measured in retinas subjected to microbead injection and rAAV2-HspB1 administration according to embodiments disclosed herein.

FIG. 16 is a confocal microscopy image panel showing the effects of intravitreal rAAV2-HspB1 administration on retinal gliosis according to embodiments disclosed herein.

### DETAILED DESCRIPTION

This disclosure relates to compositions, methods, and systems for treating, reducing the risk of, preventing, and/or alleviating at least one symptom of a retinal disease, injury, or condition, including glaucoma and associated ocular damage. Embodiments involve reducing or preventing RGC death via gene therapy approaches that involve administering a pharmaceutical composition containing an rAAV vector that encodes at least one HSP, such as Hsp27. To increase HSP levels in RGCs, specifically, the rAAV vector may include an RGC-specific promoter sequence operably linked to the HSP sequence. The pharmaceutical composition, which may also include an acceptable carrier and/or excipient, can be administered one or more times before and/or after a subject is diagnosed with an ocular condition, such as glaucoma (e.g., normal tension glaucoma), or after a subject sustains an eye injury. In some examples, only one administration of the pharmaceutical composition may suffice to effectively treat or prevent an ocular condition. Administration of the pharmaceutical composition in the manner disclosed, e.g., intravitreally, may increase the levels of anti-apoptotic HSPs in the eye, thereby significantly inhibiting RGC death that would otherwise occur after the injury or onset of an ocular condition. The rAAV vectors utilized pursuant to the gene therapies described herein may advantageously exhibit low immunogenicity and minimal cytotoxicity. Altogether, these benefits may prevent, reduce, and/or slow RGC death in a safe, effective manner not previously contemplated in the field of ocular therapy.

Unless defined otherwise below, technical and scientific terms used herein have the same meaning as commonly understood by one of ordinary skill in the art to which this invention pertains. For the purposes of the present invention, the following terms are defined for clarity.

As used herein, HSPs are stress proteins each having a crystallin core domain ranging from about 80 to about 100 amino acid residues. Among additional physiological functions, HSPs may exhibit anti-apoptotic and molecular chaperone activity within the cells in which they are present. HSPs can be divided categorically into small HSPs (˜12-43 kDa) and large HSPs (˜100-110 kDa). Examples of small HSPs include Hsp27 (also named HspB1), Hsp20 (also named HspB6), and α-crystallin (which is comprised of two subunits, αA and αB). Large HSPs include Hsp90, for example. In the present disclosure, an HSP gene or sequence comprises a nucleic acid sequence encoding an HSP protein or portion thereof.

As used herein, “subject” means a human or other mammal. Non-human subjects may include, but are not limited to, various mammals such as domestic pets and/or livestock, for example. A subject can be considered in need of treatment. The disclosed compositions, methods, and systems may be effective to treat healthy human subjects, patients diagnosed with glaucoma, patients diagnosed with one or more other ocular diseases, patients suffering from various eye injuries, diabetic patients, or patients experiencing loss of eyesight.

As used herein, an “ocular condition” encompasses all diseases or conditions related to the eye, including those that negatively affect one or both eyes of a subject. Ocular diseases, injuries, and conditions targeted by the therapeutic methods disclosed herein may damage retinal tissue specifically. Non-limiting examples of ocular conditions contemplated herein may include glaucoma, normal tension glaucoma, macular degeneration, diabetic eye disease, diabetic retinopathy, retinal gliosis, retinal detachment, retinitis pigmentosa, RGC death, elevated intraocular pressure, excitotoxic damage, physical damage (e.g., ischemia and/or reperfusion), chemical damage, neurotrophic factor deprivation, oxidative stress, inflammation, mitochondrial dysfunction, axonal transport failure, or combinations thereof.

As used herein “glaucoma” refers to a disease characterized by the permanent loss of visual function due to irreversible damage to the optic nerve. The two main types of glaucoma are primary open angle glaucoma and angle closure glaucoma, one or both of which may be treated according to embodiments described herein.

As used herein, the term “intraocular pressure” refers to the pressure of the fluid inside the eye. The intraocular pressure of a normal human eye typically ranges from about 10 to about 21 mm Hg. “Elevated” intraocular pressure is conventionally considered to be greater than or equal to about 21 mm Hg. Elevated intraocular pressure can be a risk factor for the development of glaucoma.

Treating retinal damage, as contemplated herein, encompasses treating, reducing the risk of, preventing, or alleviating at least one symptom of retinal damage caused by or associated with a disease, injury, or other condition. Accordingly, “treating,” “treatment,” or “alleviation” refers to both therapeutic treatment and prophylactic or preventative measures, wherein the object is to prevent or slow down (lessen) the targeted pathological condition and/or symptom. Those in need of treatment include those already diagnosed with the condition, as well as those prone to contracting or developing the condition. A subject is successfully “treated” for retinal damage if, after receiving a therapeutically effective amount of a pharmaceutical composition according to methods of this disclosure, the subject shows observable and/or measurable reduction in, or absence of, one or more of eyesight impairment, eyesight loss, eyesight abnormalities, RGC axonal degeneration, damage to the somas of RGCs, and RGC death. The terms “treat” or “treating” are used consistently herein for ease of illustration, only, and thus should not be construed as limiting.

“Reducing,” “reduce,” or “reduction” means decreasing the severity, scope, frequency, or length of retinal damage.

An “effective amount” of a composition containing an rAAV vector is an amount sufficient to carry out a specifically stated purpose, and may be determined empirically and in a routine manner, in relation to the stated purpose. For example, an “effective amount” as used herein can be defined as an amount of an rAAV vector that will increase or enhance HSP protein production in the RGCs of a subject. The term “therapeutically effective amount” refers to an amount of a composition containing an rAAV vector that will detectably and repeatedly treat, reduce the risk of, prevent, or alleviate at least one symptom of a retinal disease, injury, or condition in a subject. This includes, but is not limited to, a reduction in the frequency or severity of the signs or symptoms of a disease, such as elevated intraocular pressure, RGC soma damage, RGC death, vision loss, and/or RGC axonal degeneration. Such improvements may be considered relative to an eye or a subject not administered a disclosed pharmaceutical composition according to the methods disclosed herein. One skilled in the art understands that a treatment may improve a disease condition, but may not be a complete cure for the disease. For example, successful treatment of a patient with glaucoma can be evidenced by no further progression of visual field loss in the affected eye, or a slowing of the rate of progression of visual field loss in the affected eye.

“Administration of” and “administering a” compound, composition, or agent should be understood to mean providing a compound, composition, or agent, a prodrug of a compound, composition, or agent, or a pharmaceutical composition as described herein. The compound, agent or composition can be provided or administered by another person to the subject (e.g., intravitreally or intraperitoneally) or it can be self-administered by the subject.

“Pharmaceutical compositions” or “pharmaceutical formulations” are compositions that include an amount (for example, a unit dosage) of one or more of the disclosed compounds, e.g., rAAV-HSPs, together with one or more non-toxic pharmaceutically acceptable additives, including carriers, diluents, and/or adjuvants, and optionally other biologically active ingredients. Such pharmaceutical compositions can be prepared by standard pharmaceutical formulation techniques such as those disclosed in Remington's Pharmaceutical Sciences, Mack Publishing Co., Easton, Pa. (19th Edition).

As used herein, a “pharmaceutically acceptable excipient” or a “pharmaceutically acceptable carrier” means a pharmaceutically acceptable material, composition, or vehicle that contributes to the desired form or consistency of the pharmaceutical composition. Each excipient or carrier must be compatible with other ingredients of the pharmaceutical composition when comingled such that interactions which would substantially reduce the efficacy of the compositions of this disclosure when administered to a subject and interactions which would result in pharmaceutical compositions that are not pharmaceutically acceptable are avoided. In addition, each excipient or carrier must be of sufficiently high purity to render it pharmaceutically acceptable. Non-limiting examples of pharmaceutically acceptable carriers can include lactose, dextrose, sucrose, sorbitol, mannitol, starch, acacia gum, calcium phosphate, alginate, gelatin, calcium silicate, microcrystalline cellulose, polyvinyl pyrrolidone, cellulose, water, syrup, methyl cellulose, methylhydroxybenzoate, propylhydroxybenzoate, talc, magnesium stearate, mineral oil or the like. In addition or alternatively, a carrier can include comprise a lubricant, a wetting agent, a flavor, an emulsifier, a suspending agent, a preservative, or the like.

As used herein, the term “adenovirus” refers to a non-enveloped, single-stranded DNA virus. The term “recombinant adeno-associated viral vector” or “rAAV vector” refers to a recombinant adenovirus construct that includes at least one “HSP sequence,” which refers to a nucleic acid sequence, e.g., a gene, encoding an HSP, an HSP fragment, or an HSP domain. Additional nucleic acid sequences necessary for gene expression and DNA replication may be included in a given rAAV vector. Such sequences can include a tissue-specific promoter operably linked to the HSP sequence, along with one or more polyadenylation sequences, origins of replication, transcription enhancers, etc. The disclosed rAAV vectors can be introduced into a target cell with high transduction efficiency, where the vector expresses at least one HSP construct.

As used herein, the term “operably linked” refers to an arrangement of elements that allows the elements to perform their usual function. For example, a DNA coding sequence, such as an HSP sequence, can be fused to a promoter, an enhancer, and/or a terminator sequence or the like so that the coding sequence is correctly transcribed into mRNA, spliced/joined, translated into a polypeptide, and folded into the conformation necessary for the resulting protein to properly function in a living cell. The promoter and/or additional regulatory elements may not necessarily be contiguous with an HSP sequence, as long as such elements direct the expression thereof. For example, transcribed DNA sequences can be present between the promoter sequence and the HSP sequence, and the promoter sequence can still be considered “operably linked” to the coding sequence. The operable linkage to a recombinant vector may be prepared using a genetic recombination technique known in the art, such as homologous recombination.

The rAAV vectors disclosed herein can include a promoter that is heterologous, tissue-specific, constitutive or inducible. Embodiments include an RGC-specific promoter that induces robust expression of one or more nucleic acids encoding at least one HSP specifically in RGCs. In some embodiments, an RGC-specific promoter may drive expression exclusively in RGCs, thereby minimizing or eliminating potential off-target effects. In some embodiments, the promoter may comprise a human-DNA, mini-RGC-specific neuro filament light chain promoter, e.g., Ple345-NEFL. Because RGC loss is a primary driver of blindness in subjects afflicted with glaucoma, the RGC-specific promoter may be important for preventing RGC loss in subjects at risk of developing or already diagnosed with the disease.

The disclosed gene therapies and associated systems utilize pharmaceutical compositions containing expression constructs in the form of rAAV vectors formulated to induce the expression of HSPs in RGCs. As used herein, an “expression construct” may refer to any type of genetic construct comprising a nucleic acid encoding an HSP or peptide fragment thereof The expression construct may drive upregulation or over expression of the HSP(s) encoded therein.

As used herein, the terms “identity” or “similarity” denote relationships between two or more nucleic acid sequences or polypeptide sequences, as determined by comparing the sequences. In the art, identity also means the degree of sequence relatedness between polypeptide or polynucleotide sequences, as determined by the match between strings of such sequences.

The singular terms “a,” “an,” and “the” include plural referents unless context clearly indicates otherwise. Similarly, the word “or” is intended to include “and” unless the context clearly indicates otherwise. The term “comprises” means “includes.” Also, “comprising A or B” means including A or B, or A and B, unless the context clearly indicates otherwise. Although methods and materials similar or equivalent to those described herein can be used in the practice or testing of this disclosure, suitable methods and materials are described below. In addition, the materials, methods, and examples are illustrative only and not intended to be limiting.

Unless otherwise defined, all technical and scientific terms used herein have the same meaning as commonly understood by one of ordinary skill in the art to which this disclosure belongs. In the case of conflict, the present specification, including definitions, will control. All publications, patent applications, patents, and other references mentioned herein are incorporated by reference. The references cited herein are not admitted to be prior art to the claimed invention.

### Vectors

Vectors of this disclosure include rAAV vectors encoding at least one HSP, along with an RGC-specific promoter sequence.

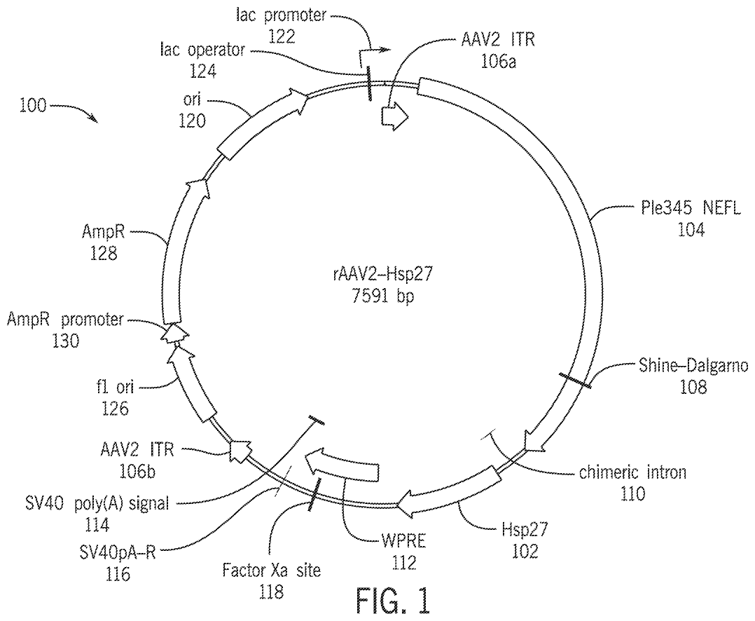

FIG. 1 illustrates an example rAAV vector 100 having SEQ ID NO: 1 utilized according to embodiments herein. As shown, the rAAV vector 100 includes an Hsp27 coding sequence 102 (“Hsp27 sequence”) positioned downstream of an RGC-specific promoter 104, which in this example is Ple345 NEFL. In some examples, Hsp27 may be the most effective HSP for preventing, reducing, and/or slowing RGC death associated with ocular injury or disease. Additional HSPs, including Hsp20, αA-crystallin, and/or αB-crystallin, may also effectively combat RGC death when delivered to a subject via an rAAV vector disclosed herein. Such HSPs may be less effective than Hsp27 in some examples. Particular HSPs may exhibit varying efficacy levels depending on the subject and/or condition treated and/or the method of treatment. One or more of the aforementioned HSPs may be specifically effective for protecting the somas and/or axons of RGCs from degeneration caused by damage or disease.

Flanking the Hsp27 sequence 102 and its promoter 104 of the rAAV vector 100 are two inverted terminal repeats 106a, 106b, which facilitate viral replication and packaging. A ribosomal binding site in the form of a Shine-Dalgarno sequence 108 is also included, as is a chimeric intron sequence 110, which enhances mRNA processing and HSP expression. The rAAV vector 100 also includes a woodchuck hepatitis post-transcriptional regulatory element (“WPRE”) 112, an SV40 polyadenylation sequence 114, an SV40pA-R sequence 116, and for enzymatic restriction cleavage, a Factor Xa site 118. The rAAV vector includes an origin of replication 120, and for binding RNA polymerase during transcription, a lac promoter 122 and overlapping lac operator sequence 124 are included. An F1 origin of replication 126 facilitates packaging of ssDNA into phage particles. For in vitro selection, the illustrated rAAV vector 100 also includes an ampicillin resistance gene 128 and upstream promoter 130. The antibiotic resistance cassette may not be included in all embodiments, including embodiments of the rAAV vector formulated for mammalian injection. One or more additional DNA constructs may be included in different embodiments, and one or more of the illustrated DNA constructs may be excluded.

The particular adenovirus used to create rAAV vectors of the present invention may vary. For example, type 1, type 2, type 3, type 4, type 5 or the like may be used. In the specifically depicted embodiment, the rAAV vector is an adeno-associated virus-Type 2 vector (“rAAV2 vector”).

The Hsp27 sequence 102 can include a nucleic acid sequence encoding a wild-type Hsp27 protein. Additionally or alternatively, embodiments may include a nucleic acid sequence encoding a polypeptide constituting a portion of an HSP, e.g., a portion of Hsp27. Such sequences may be about 50%, about 60%, about 70%, about 80%, about 90%, about 95%, or about 99% identical to the nucleic acid sequence encoding wild-type Hsp27.

In addition or alternatively, an rAAV vector contemplated herein may include one or more nucleic acid sequences encoding one or more different HSPs, such as Hsp20, αA-crystallin, or αB-crystallin, just to name a few. Accordingly, a single rAAV vector may include coding sequences for one HSP or multiple HSPs.

In some examples, the rAAV vector can include a reporter gene and/or markers for screening and/or tracking purposes. Example reporter genes can include genes encoding green fluorescent protein (GFP), modified green fluorescent protein (mGFP), enhanced green fluorescent protein (EGFP), red fluorescent protein (RFP), modified red fluorescent protein (mRFP), enhanced red fluorescent protein (ERFP), blue fluorescent protein (BFP), enhanced blue fluorescent protein (EBFP), yellow fluorescent protein (YFP), enhanced yellow fluorescent protein (EYFP), cyan fluorescent protein (CFP), enhanced cyan fluorescent protein (ECFP) or the like.

### Pharmaceutical Compositions

The pharmaceutical compositions of this disclosure are suitable for treating, reducing the risk of, preventing, or alleviating at least one symptom of an ocular disease, injury, and/or condition caused by or associated with RGC death and/or retinal gliosis. Embodiments of the pharmaceutical composition can include an rAAV vector encoding at least one HSP, along with an RGC-specific promoter sequence. The pharmaceutical composition can also include a pharmaceutically acceptable carrier configured to facilitate and/or stabilize delivery of the rAAV vector to the target site(s) of a subject.

In some embodiments, a pharmaceutical composition may include a mixture of two or more distinct rAAV vectors each encoding one or more unique HSPs. For example, a pharmaceutical composition may include an rAAV vector encoding Hsp27, an rAAV vector encoding Hsp20, an rAAV vector encoding αA-crystallin, and/or an rAAV vector encoding αB-crystallin.

In embodiments, the pharmaceutical composition may include or be administered concurrently with one or more excipients. Suitable excipients may vary depending upon the particular dosage utilized. In addition, suitable excipients may be chosen for a particular function, such as the ability to facilitate the production of stable dosage forms. Excipients may also be chosen for regulatory compliance. Non-limiting excipient examples include: fillers, binders, disintegrants, lubricants, glidants, granulating agents, coating agents, wetting agents, solvents, co-solvents, suspending agents, emulsifiers, coloring agents, anticaking agents, humectants, chelating agents, plasticizers, viscosity agents, antioxidants, preservatives, stabilizers, and surfactants. The skilled artisan will appreciate that certain pharmaceutically acceptable excipients may serve more than one function and may serve alternative functions depending on how much of the excipient is present in the final composition and which other ingredients are present in the composition.

In embodiments, the rAAV vector may be administered concurrently with one or more buffering agents and/or diluents, non-limiting examples of which may include various concentrations of sodium hydroxide and sodium phosphate.

The inclusion of particular excipients and/or carriers may depend on the route of administration. For example, a preparation for parenteral administration can include a sterile aqueous solution, a non-aqueous solvent, a suspension, an emulsion, a freeze-dried preparation, and/or a suppository. Non-aqueous solvents may include propylene glycol, polyethylene glycol, vegetable oil, and/or an injectable ester. As a base for the suppository, witepsol, macrogol, tween 61, cacao butter, laurin butter, glycerogelatin or the like may be used. To increase the stability or absorption of peptides, carbohydrates such as glucose, sucrose or dextran, antioxidants such as ascorbic acid or glutathione, chelating agents, low-molecular weight proteins or other stabilizers may be used.

In some embodiments, the pharmaceutical composition may also be provided as a topical composition, for example in droplet form. Eye drops may be formulated with an aqueous or non-aqueous base also comprising one or more dispersing agents, solubilizing agents, and/or suspension agents. According to such embodiments, the concentration of the rAAV vector in the pharmaceutical composition may be greater than the concentrations utilized for intravitreal implementations.

### Therapeutic Approaches

Methods of treating an ocular condition may involve administering to an eye of a subject a therapeutically effective amount of an rAAV-HSP composition disclosed herein. The composition can be administered after an ocular injury is sustained or an ocular disease diagnosed. Embodiments can also involve administering a disclosed rAAV-HSP composition to an undiagnosed subject to prevent the subject from developing a disease or to lessen the severity of the symptoms upon disease onset. In some examples, prophylactic administration may be performed after determining that a subject is at an above-average risk of developing an ocular disease. In some embodiments, a single intravitreal administration of a disclosed pharmaceutical composition may increase the concentration of one or more HSPs in the eye to a level sufficient to reduce the RGC loss that, if left untreated, would otherwise drive irreversible retinal damage. Embodiments may increase the concentration of one or more HSPs specifically in the somas of the targeted RGCs.

While intravitreal administration may be the most effective for localized amelioration of retinal damage, the specific mode of administration may vary. Non-limiting examples of acceptable administration methods may include intraperitoneal administration, intravenous administration, intramuscular administration, subcutaneous administration or local administration.

As noted in the preceding section, the pharmaceutical composition can include, or be administered concurrently with, at least one pharmaceutically acceptable carrier. Relatedly, the pharmaceutical composition may be administered singly or in combination with other therapeutic agents, either serially or simultaneously. Such additional agents may or may not be formulated to treat the same ocular condition(s).

In some examples, only a single administration, which may include one or more doses, of a pharmaceutical composition disclosed herein may suffice to treat an ocular condition, including glaucoma and one or more symptoms thereof. The need for only one administration may avoid issues with patient noncompliance, thereby further increasing the likelihood of success. For subjects requiring more than one administration, the frequency of administration may vary. In embodiments, a pharmaceutically effective amount of the composition may be administered weekly, monthly, or yearly. The number of times the disclosed compositions are administered to a subject, along with the length of the treatment period, may depend on the severity or type of condition causing, or at risk of causing, retinal damage. For example, embodiments in which the pharmaceutical composition is administered to treat an eye injury may involve fewer discrete administrations than embodiments in which the rAAV composition is administered to treat a disease, such as glaucoma, which may require a more sustained treatment approach. The length of the treatment period may also be patient-specific and re-evaluated periodically by a physician or other health care provider. In various embodiments, a pharmaceutical composition may be administered immediately following an injury, such as within one, two, six, 12 or 24 hours after an injury. The formulations may be administered once or multiple times, for example two, three, four, five, six, seven, eight, nine, ten times, or more. In some examples, a single dose of a disclosed pharmaceutical composition can effectively treat an ocular condition for at least about 20 weeks. According to such examples, the pharmaceutical composition may be administered once every 20 weeks.

The pharmaceutical compositions disclosed herein may be administered using an injection device, such as tuberculin syringe or an IV drip device, which may be configured specifically for the purposes described herein. In some examples, the administration device may be a single-use device, which may be included in a kit that also includes a single dose of a pharmaceutical composition. Accordingly, an injection device may constitute a part of a system for treating, reducing the risk of, preventing, or alleviating at least one symptom of retinal damage.

The therapeutically effective amount of the pharmaceutical composition administered to a subject may vary. In embodiments, each intravitreal dose of a pharmaceutical composition provided to a subject may include an rAAV concentration ranging from about 2×10⁹ pfu/ml to about 1×10¹⁰ pfu/ml, or about 1×10¹⁰ viral genomes per ml (vg/ml), about 1×10¹¹ vg/ml, about 1×10¹² vg/ml, about 1×10¹³ vg/ml, about 1×10⁹ vg/eye, about 5×10⁹ vg/eye, or more, or any concentration therebetween. Dosing may depend, for example, on the condition treated, the severity of the condition, the nature of the formulation, the method of administration, the condition of the subject, the age of the subject, the weight of the subject, or combinations thereof. Dosage levels are typically sufficient to achieve a concentration at the site of action that is at least the same as a concentration that has been shown to be active in vitro, in vivo, or in tissue culture.

To accommodate multiple administration techniques and schedules, the pharmaceutical compositions disclosed herein may be prepared in a unit-dosage form or multiple-dosage form, along with a pharmaceutically acceptable carrier and/or excipient according to a method employed by those skilled in the art. Example formulations may be in the form of an aqueous or oil-based solution, a suspension, or an emulsion. For increased stability and long-term storage, the pharmaceutical compositions may be lyophilized.

The following experimental examples are provided to illustrate example embodiments of the present invention, and should not be considered limiting.

### EXAMPLES

### Example 1

To evaluate the effects of rAAV2-mediated delivery of various HSPs on retinal conditions characterized by RGC death, such as glaucoma, a mouse model of glaucoma was generated and treated with one intravitreal administration of a pharmaceutical composition disclosed herein.

Generation of the glaucoma mouse model involved anesthetizing wild-type (WT) mice and subjecting them to ischemia/reperfusion (I/R) injury by elevating the intraocular pressure from 15 mm Hg to 120 mm Hg. The elevated pressure was maintained for an hour and then reduced back to a pressure within the normal range. This procedure caused a significant amount of RGC death (>50%), which is similar to the extent of RGC death observed in glaucoma patients.

Intravitreal injection was performed using a glass pipette attached to a Hamilton syringe (Hamilton Bonaduz AG, Bonaduz, Switzerland). The eye lids were carefully parted, and the 33-gauge needle was inserted into the vitreous just behind the limbus at a 45° angle through the sclera into the vitreous body. Two microliters of solution was injected in 1 μL increments with a 30 second gap between each injection. After injection, the needle was slowly withdrawn, and the injected area was treated with a topical antibiotic.

Four distinct rAAV2 treatment vectors, differing only by the particular HSP encoded therein, were administered intravitreally to separate groups of treatment mice 4 weeks before I/R injury and evaluated for their ability to prevent, reduce, and/or slow RGC death. Uninjured contralateral eyes not subjected to I/R injury and injured contralateral eyes were used as healthy and untreated glaucoma controls, respectively.

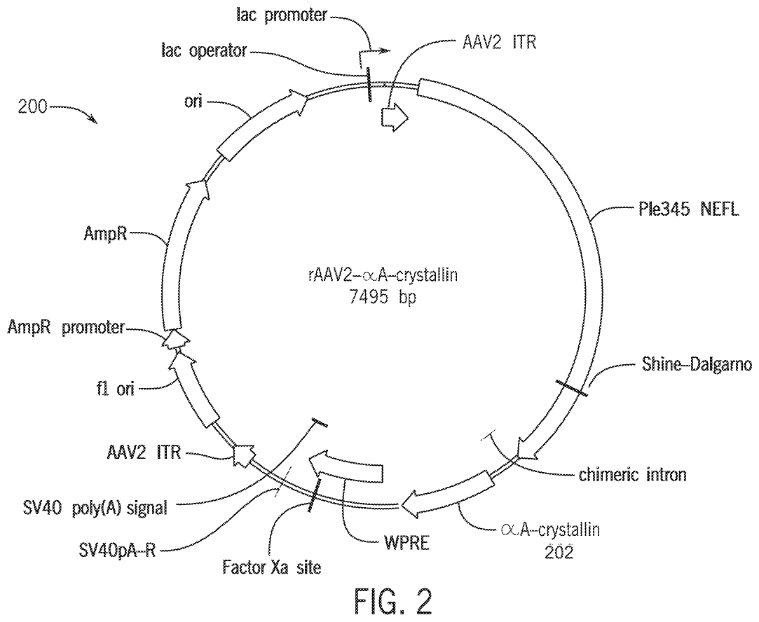

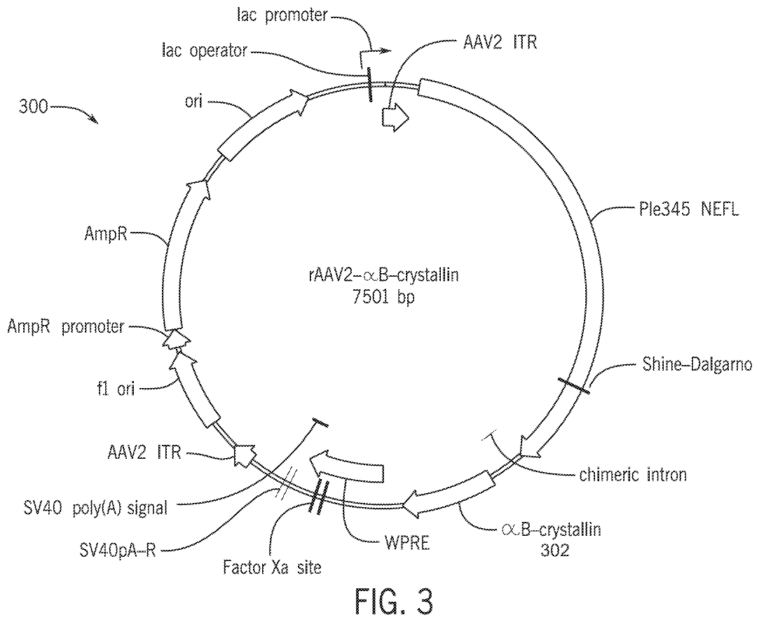

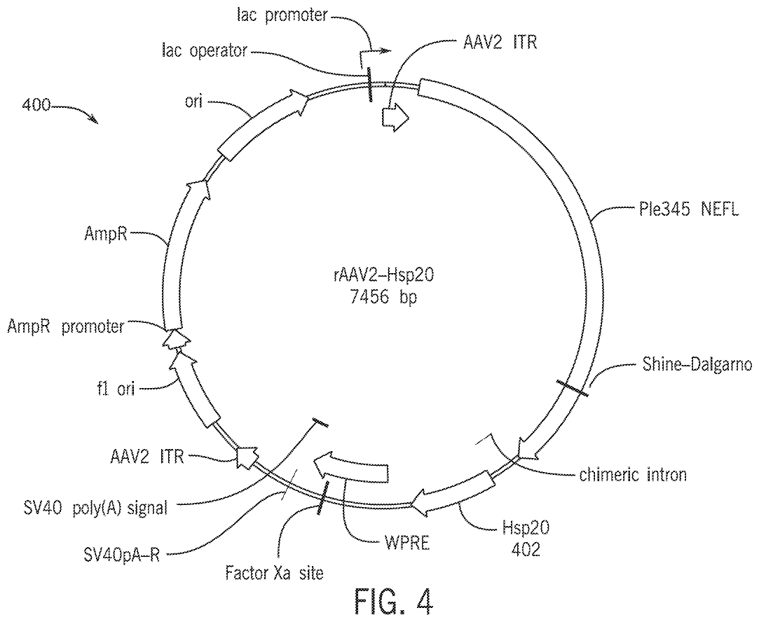

A map of one rAAV2 vector, rAAV2-HspB4, having SEQ ID NO: 2 is shown in FIG. 2. As shown, rAAV2-HspB4 200 differs from rAAV2-Hsp27 (see FIG. 1) only by the inclusion of the HspB4 (αA-crystallin) coding sequence 202, instead of the Hsp27 sequence 102. FIG. 3 shows a map of another rAAV2 vector, rAAV2-HspB5, having SEQ ID NO: 3, which includes an HspB5 (αB-crystallin) coding sequence 302 instead of the Hsp27 sequence 102. FIG. 4 shows a map of a third rAAV2 vector, rAAV2-HspB6, having SEQ ID NO: 4, which includes an HspB6 (Hsp20) coding sequence 402 instead of the Hsp27 sequence 102. The final vector, rAAV2-HspB1, comprises SEQ ID NO: 1 and is identical to rAAV2-Hsp27, as shown in FIG. 1 (Hsp27=HspB1).

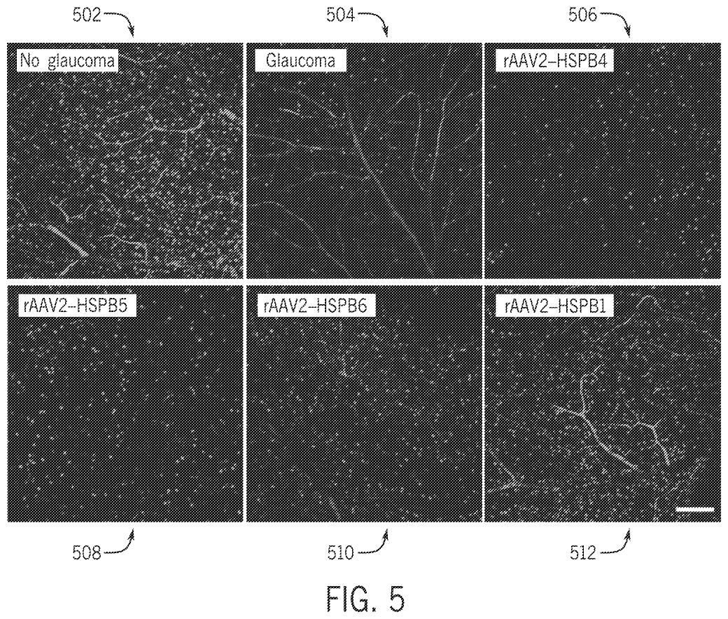

Fourteen days after I/R injury and at 6-weeks post-rAAV2 administration, all mice were anesthetized and the retinas dissected, flat-mounted and immunostained for Brn3a (brain-specific homeobox/POU domain protein 3A), which is a marker for RGCs. The effects of one-time intravitreal rAAV2 administration on RGC soma damage and total RGC loss are depicted in FIG. 5. As evidenced by greater Brn3a-positive RGC staining (lower right scale bar=100 μm), RGC numbers were the highest in the contralateral retinas removed from the healthy treatment group 502 and the lowest in the contralateral retinas removed from the untreated glaucoma group 504. Relative to the healthy mice, the rAAV2-HspB1 treatment group 512 included the highest RGC count, followed by the rAAV2-HspB6 treatment group 510, the rAAV2-HspB5 treatment group 508, and the rAAV2-HspB4 treatment group 506. Accordingly, rAAV2-HspB1 (rAAV2-Hsp27) was the most effective at reducing RGC loss in a mouse model of glaucoma. Notably, the other HSP-encoding rAAV2 vectors also reduced RGC loss relative to the untreated glaucoma mice, indicating their potential efficacy for treating ocular conditions alone or in combination.

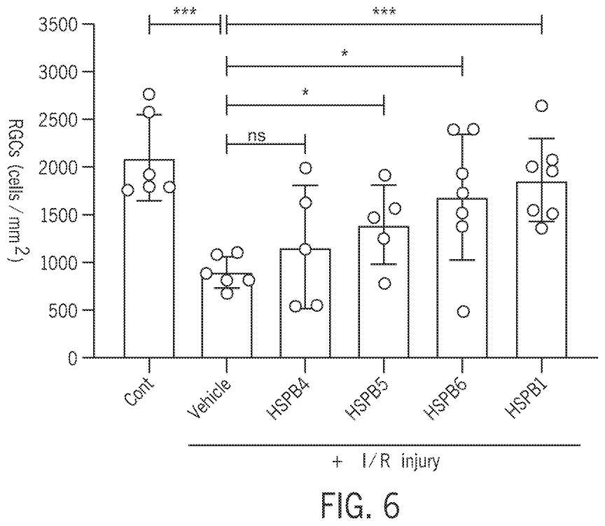

The quantitative effects of one-time, intravitreal administration of each rAAV2 vector on RGC loss, based on the number of RGCs present per square millimeter of excised retina, are shown graphically in FIG. 6, in which ns=not significant, *p<0.05 and ***p<0.001. As shown, the healthy control mice (“Cont”) not subjected to UR injury had over 2,000 RGCs per mm², whereas untreated glaucoma mice (“Vehicle”) exhibited significant RGC loss. Relative to the untreated glaucoma mice, the glaucoma mice treated with rAAV2-HspB5, rAAV2-HspB6, and rAAV2-HspB1 all exhibited statistically significant prevention of RGC loss. While not statistically significant in this study, prevention of RGC loss was also observed in the glaucoma mice treated with rAAV2-HspB4.

In view of the data obtained using the mouse model of glaucoma, only a single intravitreal administration of an rAAV2 vector encoding an HSP may be effective to significantly protect RGCs from degeneration and reduce RGC loss, with rAAV2-HspB1 (or rAAV2-Hsp27) being the most effective. The protective effect of rAAV2-HSP administration may be especially pronounced in RGC somas, as opposed to axons, although both RGC somas and axons may be protected by administration of the rAAV2 vectors disclosed herein. The disclosed rAAV2 vectors may thus exhibit long-term effects capable of preventing or at least reducing vision loss in mammals, e.g., humans, suffering from an ocular injury or disease, such as glaucoma.

### Example 2

To confirm whether the rAAV2 vectors evaluated in Example 1 effectively permeate RGCs and induce sustained overexpression of at least one HSP therein, retinal sections obtained from the untreated mice and mice treated with one of the tested rAAV2 vectors were immunostained for the administered HSP one month after intravitreal injection. The targeted RGCs were also digested one month after intravitreal administration and HSP protein levels determined via Western blotting performed using antibodies specific to each HSP.

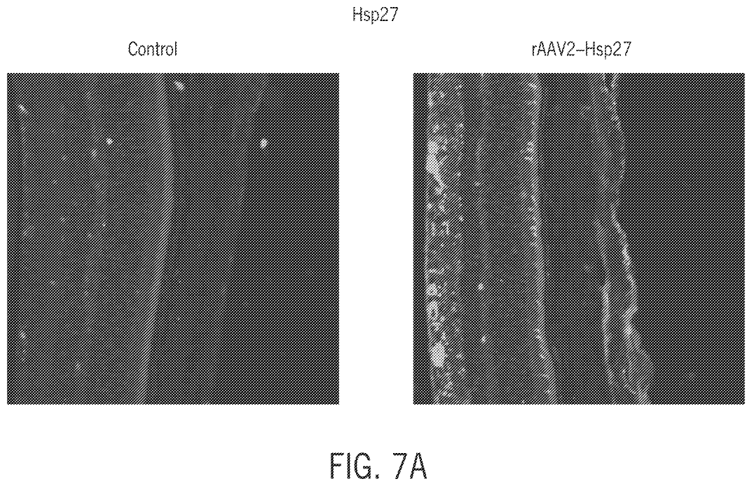

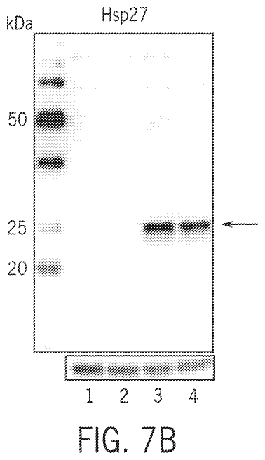

As shown in FIG. 7A, Hsp27 was over-expressed in the RGCs of retinas intravitreally injected with rAAV2-Hsp27 relative to control RGCs obtained from contralateral eyes not administered rAAV2-Hsp27. As shown graphically in FIG. 7B, duplicate samples of control cells not infected with an rAAV2 vector (lanes 1 and 2) did not include any detectable Hsp27 protein, while duplicate samples of cells infected with rAAV2-Hsp27 included robust levels of Hsp27, indicated by the thick, ˜25 kDa bands present in lanes 3 and 4.

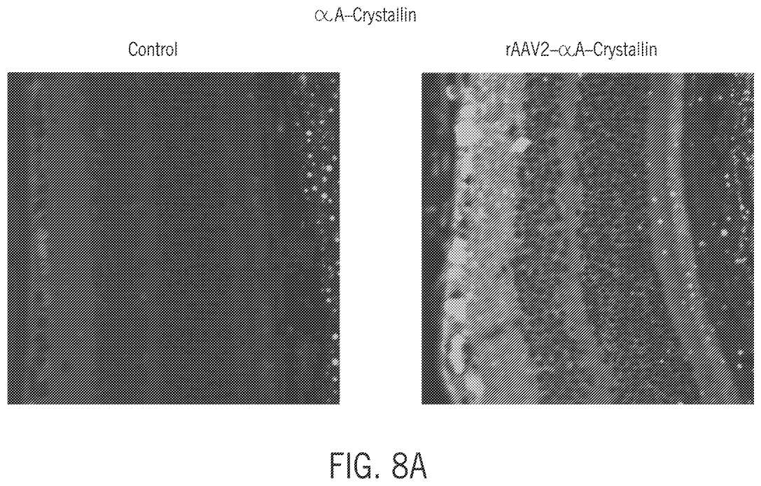

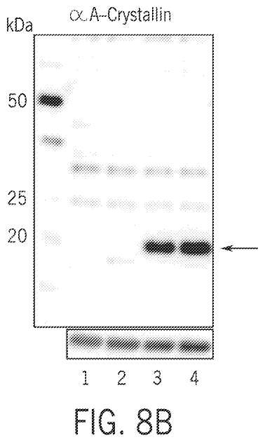

FIG. 8A shows that αA-crystallin was over-expressed in the RGCs of retinas intravitreally injected with rAAV2-αA-crystallin relative to control RGCs obtained from contralateral eyes not administered rAAV2-αA-crystallin. As shown in FIG. 8B, duplicate samples of RGCs infected with rAAV2-αA-crystallin produced robust levels of αA-crystallin, indicated by the thick, ˜19 kDa bands present in lanes 3 and 4. Lanes 1 and 2 show that control cells not infected with an rAAV2 vector did not include any detectable αA-crystallin protein.

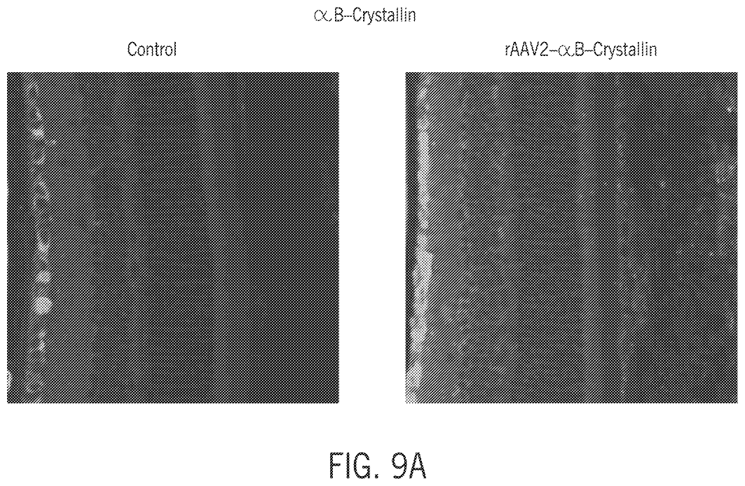

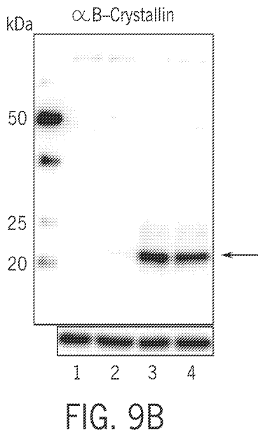

FIG. 9A shows that αB-crystallin was over-expressed in the RGCs of retinas intravitreally injected with rAAV2-αB-crystallin relative to control RGCs obtained from contralateral eyes not administered rAAV2-αB-crystallin. The blots of FIG. 9B show that RGCs infected with rAAV2-αB-crystallin produced robust levels of αB-crystallin, indicated by the thick, ˜20 kDa bands present in lanes 3 and 4. Lanes 1 and 2 show that control cells not infected with an rAAV2 vector did not include any detectable αB-crystallin protein.

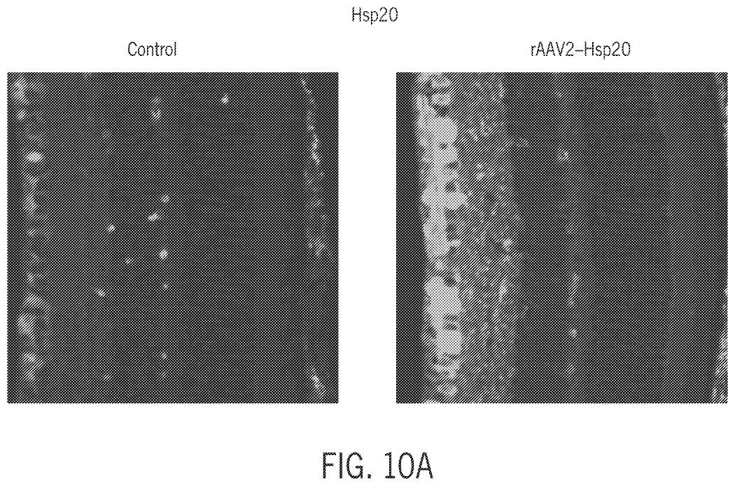

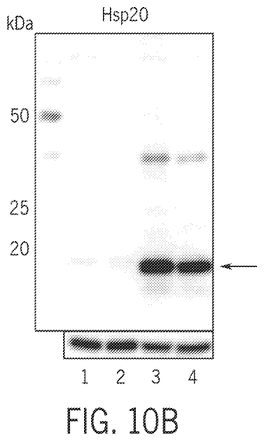

FIG. 10A shows that Hsp20 was also over-expressed in the RGCs of retinas intravitreally injected with rAAV2-Hsp20 relative to control RGCs obtained from contralateral eyes not administered rAAV2-Hsp20. The blots of FIG. 10B show that RGCs infected with rAAV2-Hsp20 produced robust levels of Hsp20, indicated by the thick, ˜18 kDa bands present in lanes 3 and 4. Lanes 1 and 2 show that control cells not infected with an rAAV2 vector did not include any detectable Hsp20 protein.

### Example 3

To determine whether rAAV2-HspB1 can prevent RGC death, a mouse model of ocular hypertension was adopted in which mice were given an intravitreal injection of the vector before intraocular pressure was increased.

In the first experiment testing the preventative impact of rAAV2-HspB1 administration, a single dose of either 1×10⁹ vg/eye or 5×10⁹ vg/eye of rAAV2-HspB1 in HBSS was injected intravitreally into separate groups of treatment mice two weeks before ocular hypertension was induced in the test animals. Two weeks later, the ocular hypertension model was generated by first anesthetizing mice via intraperitoneal injection of ketamine/xylazine supplemented with a topical application of 0.5% proparacaine hydrochloride. Ocular hypertension was induced unilaterally by injection of polystyrene microbeads (10 μm diameter, 5 million beads/mL of PBS) into the anterior chamber of the right eye of each animal. The cornea was gently punctured near the center using a 33G needle, and a small air bubble was injected to lift the anterior chamber of the eye. A small volume (2 μL) of microbeads was injected into the anterior chamber under the bubble via a micropipette connected to a Hamilton syringe. An antibiotic ointment was applied topically on the injected eye to prevent infection.

The intraocular pressure was measured weekly for four weeks using a tonometer. In particular, the mice were placed in an anesthetic chamber filled with a sustained flow of isoflurane (5% isoflurane at 2 L/minute mixed with oxygen). The tonometer took five measurements for each weekly check-in, eliminated the high and low readings, and generated an average intraocular pressure from the remaining readings for each mouse.

Four weeks after microbead injection, the eyes were dissected out and post-fixed with 4% PFA overnight at 4° C. The retinas were subsequently dissected out and washed three times in PBS before blocking (5% normal donkey serum and 1% Triton X-100 in PBS) overnight. Whole-mount retinas were then immunostained for Brn3a, which is a maker for RGCs. The Brn3a-positive RGC numbers were counted (cells/mm²) in the mid-peripheral regions from four quadrants of the whole-mounted retina using the ImageJ software (NIH). Contralateral uninjured eyes were used as a control.

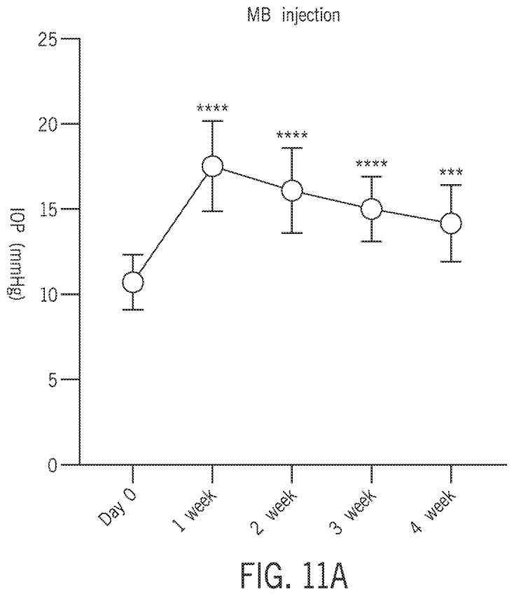

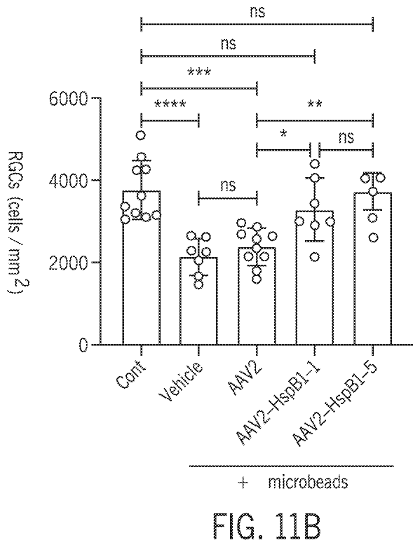

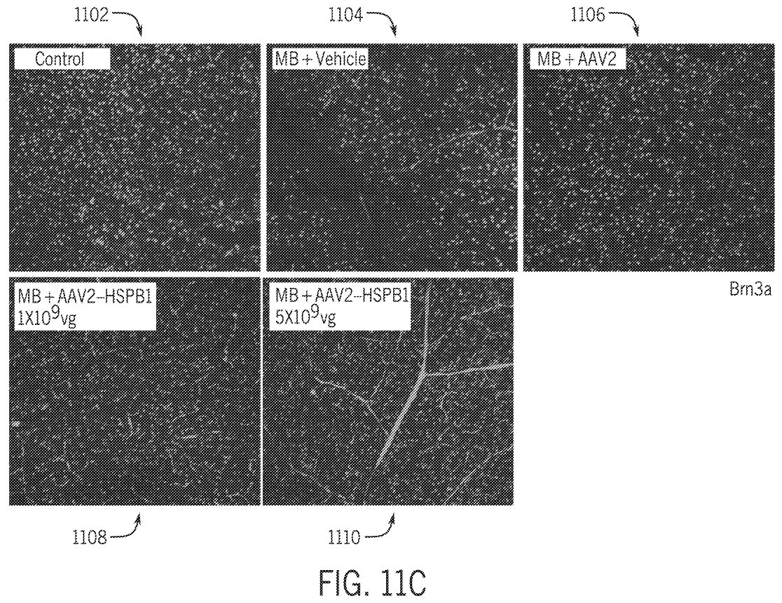

As shown in FIG. 11A, ocular microbead injection elevated the intraocular pressure from about 11 mmHg to about 18 mmHg in one week. The intraocular pressure declined thereafter, reaching a low of about 14 mmHg four weeks after microbead injection. Intraocular pressures in the hypertension mice were significantly higher than in the control mice not injected with microbeads (***p<0.001, ****p<0.0001, compared to Day 0).

The impact of intravitreally administered rAAV2-HspB1 on RGC survival six weeks after rAAV2-HspB1 injection is depicted graphically in FIG. 11B, in which ns=not significant, *p<0.05, **p<0.01, ***p<0.001, ****p<0.0001. As shown, retinas removed from the control samples not subjected to elevated intraocular pressure or treated with a viral vector had almost 3,900 RGCs per mm². By contrast, the untreated hypertension mice injected with microbeads and PBS (“Vehicle”) had only about 2,000 RGCs per mm² after six weeks, and the mice injected with microbeads and an rAAV2 capsid (“AAV2”) had about 2,300 RGCs per mm². The retinas extracted from mice treated with 1×10⁹ vg/eye of rAAV2-HspB1 retained over 3,600 RGCs per mm² after six weeks, and the retinas extracted from mice treated with 5×10⁹ vg/eye of rAAV2-HspB1 had about 3,900 RGCs per mm². Accordingly, both doses of rAAV2-HspB1 significantly prevented the loss of RGCs present within retinas having intraocular pressure elevated to hypertension levels.

Confocal microscopy images of the retinas from which the quantitative RGC concentrations of FIG. 11B were obtained are shown in FIG. 11C. As evidenced by greater Brn3a staining, RGC numbers were the highest in the retinas removed from the healthy control group 1102. Brn3a staining was much lower in the untreated ocular hypertension group 1104 and the rAAV2-capsid ocular hypertension group 1106. The 1×10⁹ vg/eye rAAV2-HspB1 group 1108 and the 5×10⁹ vg/eye rAAV2-HspB1 group 1110 showed significant prevention of RGC death relative to the untreated ocular hypertension group 1104 and the rAAV2-capsid ocular hypertension group 1106, as evidenced by greater Brn3a staining.

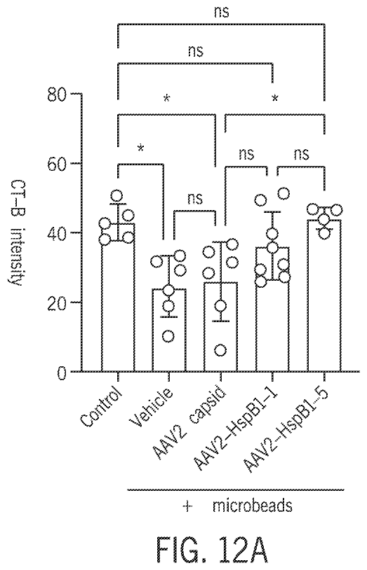

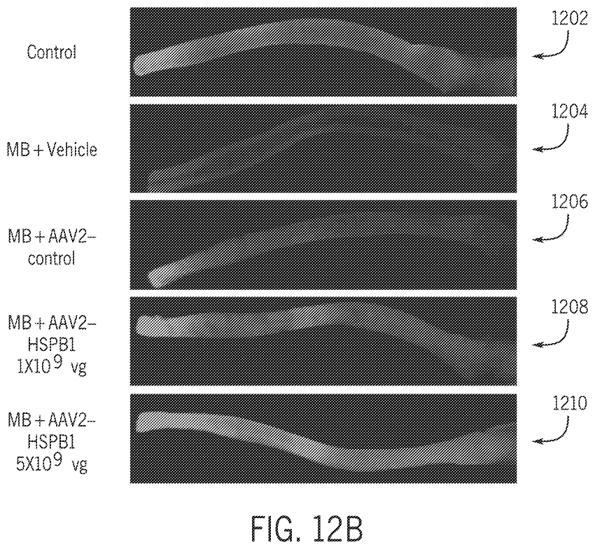

The preventative impact of rAAV2-HspB1 on axonal transport defects in RGCs was also measured six weeks after intravitreal rAAV2-HspB1 injection. As depicted graphically in FIG. 12A (ns=not significant and *p<0.05), cholera toxin B (“CT-B”) labeling was used to visualize and quantify axonal transport in RGCs. The retinas removed from the control mice exhibited CT-B intensity values of about 42 six weeks after rAAV2-HspB1 injection. By contrast, the untreated mice injected with microbeads in PBS (“Vehicle”) exhibited CT-B intensity values of only about 25 after six weeks, and the mice injected with microbeads and an rAAV2 capsid exhibited CT-B intensity values of about 26. The retinas extracted from mice treated with 1×10⁹ vg/eye of rAAV2-HspB1 exhibited CT-B intensity values of about 35 after six weeks, and the retinas extracted from mice treated with 5×10⁹ vg/eye of rAAV2-HspB1 exhibited CT-B intensity values of about 42. Accordingly, the lower dose of rAAV2-HspB1 partially prevented the development of axonal transport defects within RGCs subjected to intraocular pressure elevation, while the greater dose of rAAV2-HspB1 significantly prevented the development of axonal transport defects. Axonal transport within the RGCs injected with 5×10⁹ vg/eye of rAAV2-HspB1 was approximately equal to the axonal transport measured in RGCs not subjected to elevated intraocular pressure, indicating that intravitreal administration of 5×10⁹ vg/eye of rAAV2-HspB1 may be sufficient to substantially prevent the development of axonal transport defects in mice later afflicted with ocular hypertension.

Confocal microscopy images of the RGC axons from which the quantitative CT-B intensity values of FIG. 12A were obtained are shown in FIG. 12B. As evidenced by relatively high CT-B staining, axonal transport in the healthy control group 1202 was similar to the axonal transport measured in the hypertension group injected with 5×10⁹ vg/eye of rAAV2-HspB1 1210. Axonal transport was noticeably lower in the untreated ocular hypertension group 1204 and in the rAAV2-capsid hypertension group 1206. The 1×10⁹ vg/eye rAAV2-HspB1 group 1208 group showed a degree of axonal transport preservation relative to the untreated ocular hypertension group 1204 and the rAAV2-capsid hypertension group 1206. Accordingly, the reduction in axon-mediated CT-B transport caused by ocular hypertension was substantially prevented via intravitreal administration of 5×10⁹ vg/eye rAAV2-HspB1.

### Example 4

To determine whether rAAV2-HspB1 intervention following intraocular pressure elevation can reduce, eliminate, or slow RGC death and axonal transport defects, a mouse model of ocular hypertension was adopted in which mice were given intravitreal injections of the vector after intraocular pressure was increased.

The ocular hypertension model was generated by initially anesthetizing mice via intraperitoneal injection of ketamine/xylazine supplemented with a topical application of 0.5% proparacaine hydrochloride. Ocular hypertension was induced unilaterally by injection of polystyrene microbeads (10 μm diameter, 5 million beads/mL of PBS) into the anterior chamber of the right eye of each animal. The cornea was gently punctured near the center using a 33G needle, and a small air bubble was injected to lift the anterior chamber of the eye. A small volume (2 μL) of microbeads was injected into the anterior chamber under the bubble via a micropipette connected to a Hamilton syringe. An antibiotic ointment was applied topically on the injected eye to prevent infection.

The intraocular pressure was measured weekly for six weeks using a tonometer. In particular, the mice were placed in an anesthetic chamber filled with a sustained flow of isoflurane (5% isoflurane at 2 L/minute mixed with oxygen). The tonometer took five measurements for each weekly check-in, eliminated the high and low readings, and generated an average intraocular pressure from the remaining readings for each mouse.

One week after ocular hypertension was induced in the test animals, a single dose of rAAV2-HspB1 (1×10⁹ viral genomes in 1 μL Hank's balanced salt solution (HBSS)) was injected intravitreally into a treatment group of mice. As indicated by the second arrow (the first arrow represents the initial microbead injection), intraocular pressure was increased yet again via a second microbead injection 2 weeks after rAAV2-HspB1 administration.

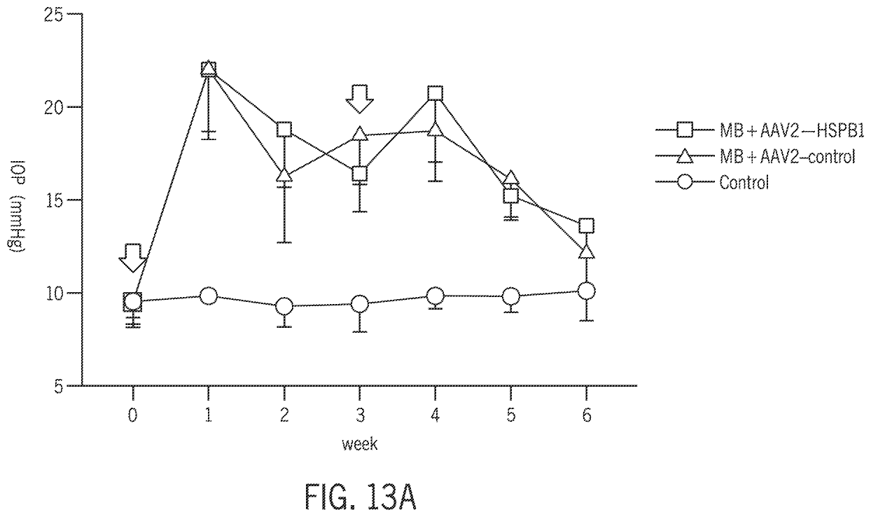

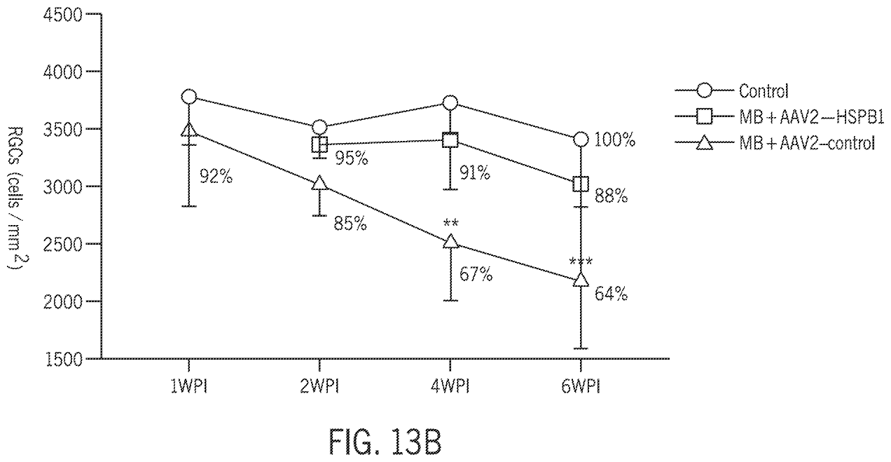

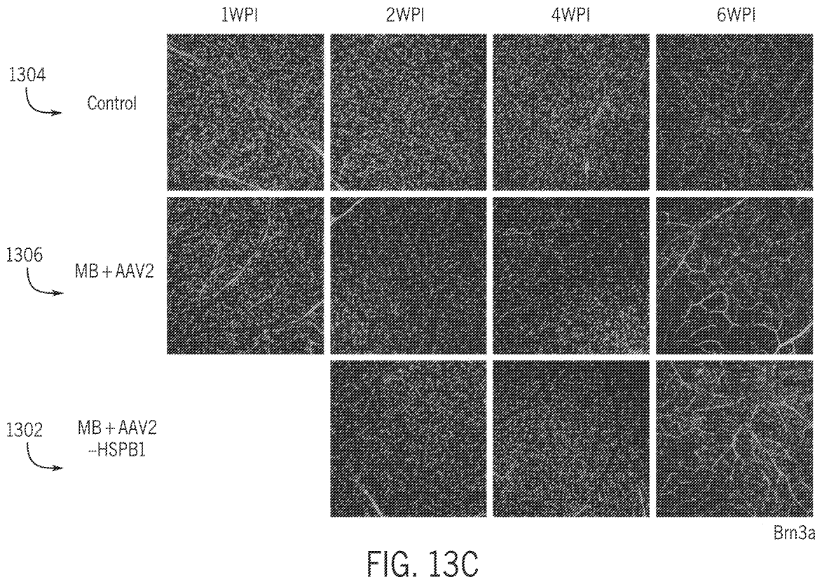

As shown in the line graph of FIG. 13A, the first ocular microbead injection elevated the intraocular pressure from about 10 mmHg to about 23 mmHg in one week in retinas subjected to microbead and rAAV2 capsid injection. The intraocular pressure declined until week 3, at which time the second microbead injection was administered. The non-injected, untreated control eyes maintained an approximately constant IOP throughout the duration of the experiment. Eyes injected with microbeads and rAAV2-HspB1 exhibited elevated IOP levels at weeks 2 and 4 relative to the heathy control and the ocular hypertension eyes injected with the AAV2 capsid. At week 3, however, intravitreal injection of rAAV2-HspB1 lowered intraocular pressure relative to the ocular hypertension eyes injected with the AAV2 capsid.

The impact of intravitreal rAAV2-HspB1 injection on RGC survival in eyes subjected to ocular pressure elevation is depicted graphically in FIG. 13B. As shown, the retinas removed from the control mice had almost 3,800 RGCs per mm² one week after the first microbead injection. The number of RGCs decreased to about 3,500 per mm² at two weeks, rose to about 3,700 per mm² at four weeks, and then dipped to about 3,400 per mm² at week 6.

By contrast, the mice injected with microbeads and an rAAV2 capsid exhibited a steady decline in the number of RGCs per mm² over the course of the study, beginning at about 3,500 per mm² at the one week mark, 3,000 per mm² at two weeks, 2,500 per mm² at four weeks, and 2,200 per mm² at six weeks.

The retinas extracted from mice treated with rAAV2-HspB1 had about 3,400 RGCs per mm² at week 2 of the study. The concentration of RGCs then rose to about 3,500 RGCs per mm² at week 4, then fell to about 3,100 RGCs per mm² at week 6. The final RGC concentration in retinas treated with rAAV2-HspB1 was therefore about 88% of the final RGC concentration measured in healthy control retinas. Accordingly, intravitreal injection of rAAV2-HspB1 significantly reduced the loss of RGCs in retinas subjected to elevated intraocular pressure relative to retinas subjected to elevated intraocular pressure and injected with an rAAV2 capsid (**p<0.01, ***p<0.001).

Confocal microscopy images of the retinas from which the quantitative RGC concentrations of FIG. 13B were obtained are shown in FIG. 13C. As evidenced by greater Brn3a staining at week 6 of the study, RGC numbers in the rAAV2-HspB1 group 1302 were comparable to the RGC numbers of the healthy control group 1304, whereas RGC numbers declined significantly in the untreated ocular hypertension group 1306 over the course of the study.

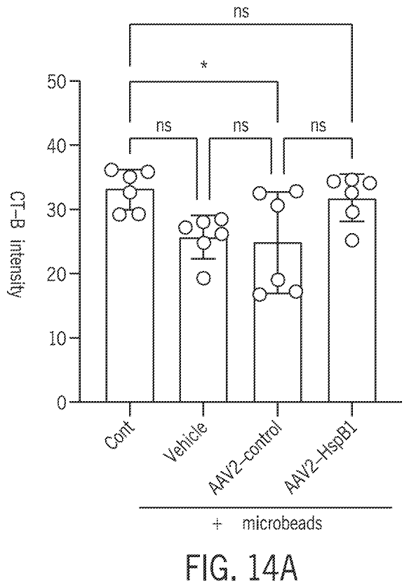

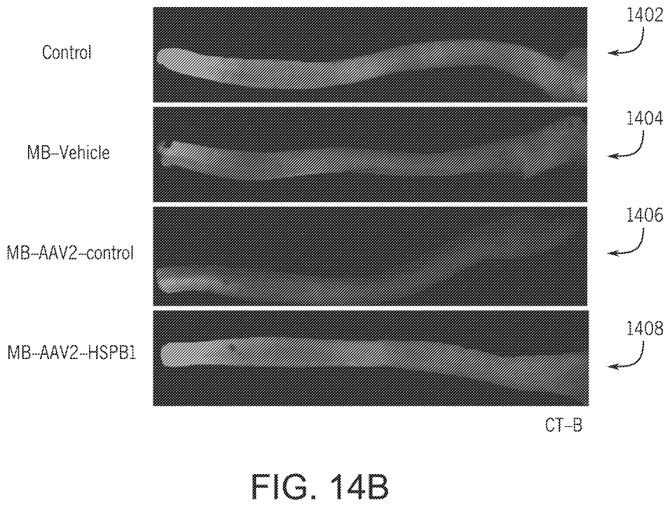

The impact of intravitreal rAAV2-HspB1 injection on axonal transport after six weeks of ocular hypertension was also measured and is depicted graphically in FIG. 14A, in which ns=not significant and *p<0.05. As shown, the retinas removed from the control mice exhibited CT-B intensity values of about 40 after six weeks of study participation. By contrast, the untreated mice injected with microbeads and PBS (“Vehicle”) exhibited CT-B intensity values of about 30 at six weeks, and the mice injected with microbeads and an rAAV2 capsid exhibited CT-B intensity values of about 29. The retinas extracted from mice treated with rAAV2-HspB1 exhibited CT-B intensity values of about 39 after six weeks. Accordingly, intravitreal rAAV2-HspB1 administration caused a reduction in axonal transport defects within RGCs subjected to intraocular pressure elevation relative to untreated RGCs subjected to the same intraocular pressure increase. Axonal transport within the RGCs injected with rAAV2-HspB1 was approximately equal to the axonal transport measured in RGCs not subjected to elevated intraocular pressure.

Confocal microscopy images of the retinas from which the quantitative CT-B intensity values of FIG. 14A were obtained are shown in FIG. 14B. As evidenced by CT-B staining, axonal transport was the greatest in the healthy control group 1402. CT-B intensity was much lower in the untreated ocular hypertension group 1404 and rAAV2-capsid ocular hypertension group 1406. The rAAV2-HspB1 group 1408 preserved approximately normal axonal transport levels relative to the untreated ocular hypertension group 1404 and rAAV2-capsid ocular hypertension group 1406.

### Example 5

To determine whether intravitreal administration of rAAV2-HspB1 can alleviate RGC function decline over a 20-week period, a mouse model of ocular hypertension was adopted in which mice were given intravitreal injections of the vector after intraocular pressure was increased.

The ocular hypertension model was generated via microbead injection at day 1 of the experiment, followed by subsequent microbead injections at weeks 3 and 6. The intraocular pressure was measured weekly for 20 weeks using a tonometer. One week after the first microbead injection, a single dose of rAAV2-HspB1 or AAV2 capsid was injected intravitreally into separate groups of mice.

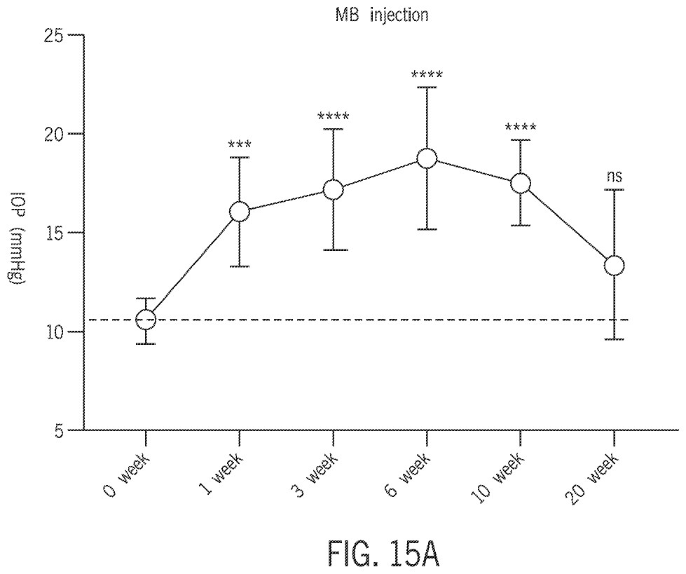

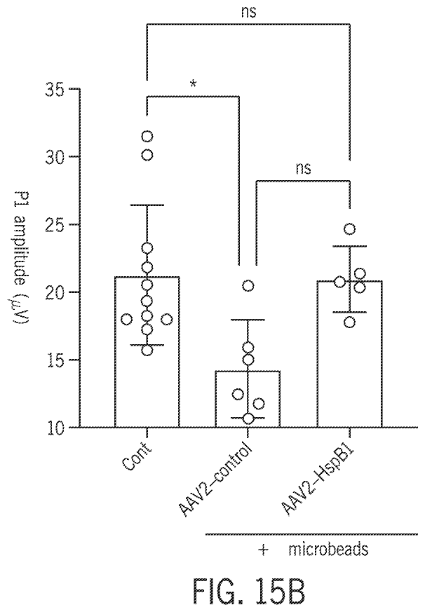

As shown in FIG. 15A, ocular microbead injection significantly elevated intraocular pressure from a starting value of about 10 mmHg to about 18 mmHg at week 6, after which the pressure dropped to about 13 at week 20 (ns=not significant, ***p<0.001, ****p<0.0001).

RGC function was assessed via pattern electroretinogram (PERG) amplitude over the 20-week duration of the study. PERG measurements were conducted using the Jörvec instrument (Intelligent Hearing Systems, Miami, FL), as per manufacturer's instructions. Reference and ground electrodes were placed subcutaneously in the scalp and at the tail region, respectively, and corneal electrodes were positioned at the lower fornix in contact with the eye globe. Small drops of GelTear eye drops were applied to both eyes to prevent corneal dryness. Two separate LED monitors attached to the system were used to display contrast-reversing horizontal bars at a spatial frequency of 0.095 cycles/degree and luminance of 500 cd/m2. The distance between the display monitors and the eyes were maintained at 10 cm. The LED monitors were placed at an angle of approximately 60 degrees for a better projection of the light signals. PERG waveforms generated for each run consisting of 372 sweeps (on-off) from both eyes were then processed and averaged by the PERG software separately for each eye. The grand average PERG waveforms were analyzed using the PERG software to identify the major positive (P1) and negative waves to calculate the amplitude and latency.

The P1 amplitude readings (measured in μV) are shown in FIG. 15B. As shown, intraocular pressure elevation in the eyes of rAAV2-caspid mice caused a significant decline in the P1 amplitude compared to the control mice. By contrast, intravitreal rAAV2-HspB1 injection sustained a P1 amplitude approximately equal to the P1 amplitude measured in the control mice. (*p<0.03; ns=not significant). Accordingly, intravitreal rAAV2-HspB1 injection improved the visual function of RGCs in a mouse model of glaucoma generated by elevating intraocular pressure.

### Example 6

To determine whether intravitreal injection of rAAV2-HspB1 can alleviate retinal gliosis, a mouse model of ocular hypertension was adopted in which mice were given an intravitreal injection of the vector after intraocular pressure was increased.

For the gliosis study, a control group of mice was not injected with microbeads or a viral vector, a positive control group was injected with microbeads and an rAAV2 capsid, and a third group was injected with microbeads and rAAV2-HspB1. Glial fibrillary acid protein (“GFAP”) and ionized calcium-binding adapter molecule 1 (“Iba1”) staining was used to identify retinal gliosis, which is evidenced by a proliferation of well-differentiated glial cells. Greater staining represented greater levels of gliosis.

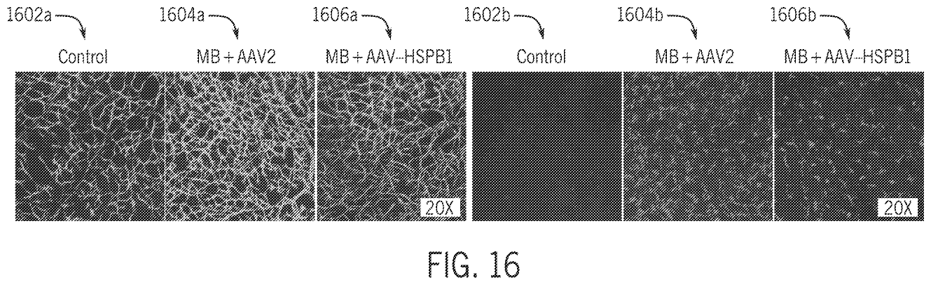

As shown in the 20× confocal microscopy images of FIG. 16, the control group 1602a,b exhibited less GFAP and Iba1 staining, respectively, relative to the untreated ocular hypertension group 1604a,b injected with an rAAV2 capsid, which exhibited a notable increase in gliosis. Gliosis was reduced in the rAAV2-HspB1-treated hypertension mice 1606a,b to a level substantially similar to the healthy control group.

Although various representative embodiments and implementations have been described above with a certain degree of particularity, those skilled in the art could make numerous alterations to the disclosed embodiments without departing from the spirit or scope of the inventive subject matter set forth in the specification and claims. In some instances, in methodologies directly or indirectly set forth herein, various steps and operations are described in one possible order of operation, but those skilled in the art will recognize that steps and operations may be rearranged, replaced, or eliminated without necessarily departing from the spirit and scope of the present disclosure. It is intended that all matter contained in the above description or shown in the accompanying drawings shall be interpreted as illustrative only and not limiting. Changes in detail or structure may be made without departing from the spirit of the disclosure as defined in the appended claims.

1. A method of treating, reducing the risk of, preventing, or alleviating at least one symptom of a retinal disease, injury, or condition in a subject, the method comprising:
administering to the subject a therapeutically effective amount of a composition comprising a recombinant adeno-associated viral vector, the vector comprising:
a nucleic acid sequence encoding at least one biologically active heat shock protein, wherein the at least one biologically active heat shock protein comprises Hsp27; and
a promoter sequence positioned upstream of the nucleic acid sequence, wherein the promoter sequence induces expression of the nucleic acid sequence in retinal ganglion cells.

2. The method of claim 1, wherein the retinal ganglion cells comprise mammalian retinal ganglion cells.

3. The method of claim 1, wherein the composition is administered at least once within 24 hours after the injury is sustained by the subject or the retinal disease or condition is diagnosed.

4. The method of claim 1, wherein the composition is administered intravitreally.

5. The method of claim 1, wherein the composition is administered only once.

6. The method of claim 1, wherein the adeno-associated viral vector comprises an adeno-associated virus-Type 2 vector.

7. The method of claim 1, wherein the retinal disease, injury, or condition is glaucoma.

8. The method of claim 1, wherein the retinal disease, injury, or condition is selected from the group consisting of: macular degeneration, diabetic eye disease, retinal detachment, and retinitis pigmentosa.

9. The method of claim 1, wherein the retinal disease, injury, or condition is caused by excitotoxic damage, physical damage, chemical damage, neurotrophic factor deprivation, oxidative stress, inflammation, mitochondrial dysfunction, axonal transport failure, or combinations thereof.

10. The method of claim 1, wherein the retinal disease, injury, or condition comprises a loss of retinal ganglion cells, an increase in intraocular pressure, or both.

11. A system for treating, reducing the risk of, preventing, or alleviating at least one symptom of a retinal disease, injury, or condition in a subject, the system comprising:
an injection device; and
a therapeutically effective amount of a composition comprising a recombinant adeno-associated viral vector, the vector comprising:
a nucleic acid sequence encoding at least one biologically active heat shock protein, wherein the at least one biologically active heat shock protein comprises Hsp27; and
a promoter sequence positioned upstream of the nucleic acid sequence, wherein the promoter sequence induces expression of the nucleic acid sequence in retinal ganglion cells,
wherein the injection device is configured to administer the composition to the subject intravitreally.

12. The system of claim 11, wherein the retinal disease, injury, or condition is glaucoma.

13. The system of claim 11, wherein the retinal disease, injury, or condition comprises a loss of retinal ganglion cells, an increase in intraocular pressure, or both.

14. The system of claim 11, wherein the retinal ganglion cells comprise mammalian retinal ganglion cells.

15. The system of claim 11, wherein the injection device is a single-use device.

16. The system of claim 11, wherein the adeno-associated viral vector comprises an adeno-associated virus-Type 2 vector.

17. A pharmaceutical composition comprising:
a recombinant adeno-associated viral vector comprising:
a nucleic acid sequence encoding at least one biologically active heat shock protein, wherein the at least one biologically active heat shock protein comprises Hsp27; and
a promoter sequence positioned upstream of the nucleic acid sequence, wherein the promoter sequence induces expression of the nucleic acid sequence in retinal ganglion cells; and
a pharmaceutically acceptable carrier,
wherein the pharmaceutical composition is formulated for treating, reducing the risk of, preventing, or alleviating at least one symptom of a retinal disease, injury, or condition in a subject.

18. The pharmaceutical composition of claim 17, wherein the pharmaceutical composition is formulated for intravitreal administration.

19. The pharmaceutical composition of claim 17, wherein the adeno-associated viral vector comprises an adeno-associated virus-Type 2 vector.

20. The pharmaceutical composition of claim 17, wherein the retinal disease, injury, or condition comprises one or more of: a loss of retinal ganglion cells, an increase in intraocular pressure, or glaucoma.
